(19) United States
(12) Patent Application Publication
(10) Pub. No.: US 2006/0129084 A1
(43) Pub. Date: Jun. 15, 2006

(54) Method of detecting disorder of syringe pump, liquid sucking discharging device, and biochemical analyzer

(75) Inventor: Takahiro Miyato, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 11/238,047

(22) Filed: Sep. 29, 2005

(30) Foreign Application Priority Data
Sep. 29, 2004 (JP) 2004-285155

(51) Int. Cl. A61N 1/30

(52) U.S. Cl. 604/19

(57) ABSTRACT

A pressure sensor is connected to an intermediate portion of an air pipe that connects a syringe pump to a spotting nozzle of a spot fixing device. The syringe pump consists of a syringe and a plunger that slides up and down in the syringe, for sucking and discharging a liquid through the spotting nozzle. While moving the plunger in the same work range as in the actual sucking discharging operation, internal pressure of the syringe pump is measured by the pressure sensor, and derivative values of the measure pressure are obtained by differentiation. The derivative values are compared with threshold values, to detect disorder of the syringe pump, such as leakage caused by insufficient air-tightness of the syringe to the plunger or some defect in the plunger.

### FIELD OF THE INVENTION

The present invention relates to a method of detecting disorder of a syringe pump, a liquid sucking discharging device and a biochemical analyzer. More particularly, the present invention relates to a liquid sucking discharging device, a biochemical analyzer, and a method of detecting disorder or malfunction of a syringe pump that is used in the liquid sucking discharging device for fixing a spot of a specimen such as blood or urine on a sampling material such as a dry type analyzing element or an electrolyte slide.

### BACKGROUND ARTS

Medical institutions and laboratories have recently been demanded to analyze an enormous number of specimens. For quantitative analysis of chemical components and the like as contained in the specimens, it becomes popular to use biochemical analyzers that carry out the quantitative analysis through colorimetry using dry type chemical analyzing elements, and/or potentiometry using electrolyte slides, or called dry type ion selection electrode filmstrips, because the dry type analyzing elements and the electrolyte slides permit the quantitative analysis of a particular chemical component or a particular formed component of a specimen just by fixing a spot of the specimen on them. For example, a desktop type biochemical analyzer has been brought into the market under a trade name Fuji Drychem 3500, that is produced by the present applicant.

The calorimetric biochemical analyzer fixes a spot of a specimen on a dry type analyzing element and then keeps the dry type analyzing element at a constant temperature for a given time in an incubator, to induce a color reaction or pigment producing reaction. Thereafter, the dry type analyzing element is illuminated with a measuring illumination light containing a light component of a predetermined wavelength, to measure optical densities of the analyzing element. A density value of a biochemical material can be determined based on the measured optical densities. On the other hand, a potentiometeric biochemical analyzer does not measure optical densities, but determines a density value of a material by potentiometeric quantitative analysis on ion activity of a particular ion contained in the specimen fixed on a pair of electrodes consisting of two dry ion selection electrodes of the same kind.

In either method, the biochemical analyzer uses a liquid sucking discharging device for fixing a spot of a specimen on a sampling material, like the dry type analyzing element or the electrolyte slide. The liquid sucking discharging device is provided with a spotting nozzle and a sucking discharging pump for supplying the spotting nozzle with a sucking pressure and a discharging pressure. The specimen is generally a liquid and contained in a sampling cup when placed in the biochemical analyzer. The spotting nozzle is movable vertically and horizontally and, if necessary, a single-use nozzle tip is fitted on a tip of the spotting nozzle. The spotting nozzle sucks the specimen from the sampling cup and then moves to a dripping position above the sampling material, to discharge the specimen a given amount to fix it as a spot on the sampling material. As a sucking discharging pump constituting the liquid sucking discharging device, a syringe pump is known, which consists of a syringe and a plunger sliding in the syringe.

In the above described biochemical analyzer, if any leak is caused by some defect such as insufficient sealing or air-tightness of the pressure system of the liquid sucking discharging device, the device cannot normally suck and discharge the specimen, which comes up with a malfunction and lowers the accuracy of measurement. In order to let the spotting nozzle suck and discharge a given amount of liquid with accuracy, it is necessary to detect leakage in the pressure system. According to Japanese Laid-open Patent Application No. 2000-258437, a pressure sensor is connected to a pressure system for a spotting nozzle of a biochemical analyzer. On detecting pressure, a tip of a spotting nozzle is closed, and a plunger of a sucking discharging pump is stopped at several points during an operation to apply a sucking pressure and a discharging pressure. The pressure sensor measures the pressure at the time points when the plunger stops.

A liquid dispensing apparatus disclosed in Japanese Laid-open Patent Application No. 2002-350453, sucks and discharges a test liquid, to judge that the dispensing apparatus is normal when a liquid level of the discharged test liquid is within a tolerance range around a normal liquid level. If the liquid level of the discharged test liquid is out of the tolerance range, the dispensing apparatus is judged to be in trouble such as leakage.

Meanwhile, as disclosed in the above mentioned Japanese Laid-open Patent Application No. 2000-258437, it is usual to detect the trouble like the leakage based on variations in pressure measured as detection signals from the pressure sensor. Since it is hard to detect a small pressure variation from the detection signal, Japanese Laid-open Patent Application No. Hei 8-114605 suggests a sampling method wherein pressure derivative values are calculated by differentiating detection signals from a pressure sensor, and are compared with a reference value, so as to detect even a small variation in pressure.

However, according to the leak detection method disclosed in the above Japanese Laid-open Patent Application No. 2000-258437, the pressure is measured while the tip of the spotting nozzle is closed, so the leak detection is carried out under different conditions from where actual sucking discharging operation is carried out. Besides, since the pressure is detected while the plunger stops, it is impossible to detect the leakage that may occur while the plunger is moving for the sucking discharging operation. Furthermore, Japanese Laid-open Patent Application No. 2000-258437 merely discloses the leakage caused by insufficient sealing of the syringe to the plunger, but does not consider such leakage as caused by some malfunction of the plunger itself.

On the other hand, according to the method disclosed in the above mentioned Japanese Laid-open Patent Application No. 2002-350453, it is necessary to suck and discharge the test liquid for the sake of detecting the leakage. This method is inefficient and costly because it uses the test liquid. Also the method disclosed in the above mentioned Japanese Laid-open Patent Application No. Hei 8-114605 has a problem that it cannot detect leakage of a syringe because the pressure is measured while a tip of the nozzle is brought into contact with the liquid, to determine the liquid surface based on the derivative value calculated from the pressure detection signal.

### SUMMARY OF THE INVENTION

In view of the foregoing, a primary object of the present invention is to provide a method of detecting malfunctions of a syringe pump of a liquid sucking discharging device, the liquid sucking discharging device, and a biochemical analyzer using the liquid sucking discharging device, which can detect malfunctions of the liquid sucking discharging device caused by some disorder in a syringe or a plunger of the syringe pump.

To achieve the above and other objects in a method of detecting disorder of a syringe pump comprising a cylindrical syringe and a plunger sliding inside the syringe, the present invention suggests comprising steps of connecting an open pipe system to a vent port of the syringe; measuring pressure in the open pipe system while moving the plunger relative to the syringe; obtaining derivative values of the measured pressure; and judging disorder of the syringe pump based on the pressure derivative values.

It is preferable to measure the pressure of the open pipe system while moving the plunger relative to the syringe in a working range used in an actual operation of the syringe pump.

According to a preferred embodiment, disorder of the syringe pump is judged by comparing the pressure derivative values with predetermined threshold values.

In order to detect disorder of a syringe pump that is connected to a spot fixing nozzle to supply sucking and discharging pressure to the spot fixing nozzle for causing the spot fixing nozzle to suck and discharge a liquid through its tip or through a nozzle tip attached to the tip of the spot fixing nozzle, to fix a spot of the liquid on a sampling material, the method of the present invention comprises steps of connecting a pressure sensor to a pressure system that is connected between the spot fixing nozzle and the syringe pump; obtaining pressure derivative values by differentiating pressure signals output from the pressure sensor while the plunger is being moved relative to the syringe; and judging disorder of the syringe pump based on the pressure derivative values.

It is preferable to open the tip of the spot fixing nozzle while pressure sensor outputs the pressure signal. It is also preferable to move the plunger relative to the syringe in a working range used in an actual operation of the syringe pump, when to obtain the pressure signals from the pressure sensor.

A liquid sucking discharging device of the present invention comprises a syringe pump comprising a cylindrical syringe and a plunger sliding inside the syringe; an open pipe system connected to a vent port of the syringe; a pressure sensor for measuring pressure in the open pipe system; a differentiation device for differentiating pressure signals output from the pressure sensor; and a judging device for judging disorder of the syringe pump base on pressure derivative values obtained by the differentiation device.

The open pipe system comprises a nozzle that sucks and discharges a liquid, and a pipe connecting the nozzle to the vent port of the syringe pump.

The nozzle can be a spot fixing nozzle that sucks and discharges a liquid through its tip or through a nozzle tip attached to the tip of the spot fixing nozzle, to fix a spot of the liquid on a sampling material. In that case, the pressure sensor is connected to a pressure system that is connected between the spot fixing nozzle and the syringe pump. It is preferable to output the pressure signal while the plunger is being moved relative to the syringe in a working range used in an actual operation of the syringe pump, with the tip of the spot fixing nozzle open.

A preferable biochemical analyzer is produced by use of the liquid sucking discharging device of the present invention.

The method of the present invention makes sure to detect any small variations in pressure based on the pressure derivative values with high accuracy, so that any malfunctions of the liquid sucking discharging device caused by some disorder in the syringe or the plunger of the syringe pump may be detected without fail.

Because the disorder of the syringe pump is detected based the pressure that is measured while moving the plunger of the syringe pump in the same work range as in the actual sucking discharging operation, and with the tip of the spotting nozzle open, like in the actual operation, it becomes possible to detect such disorders or malfunctions that can occur during the actual sucking discharging operation.

By comparing the pressure derivative values with a threshold value that is predetermined to detect the defective sealing of the syringe, and a second threshold value that is predetermined to detect the defect in the plunger such as leaning of the plunger or insufficient roundness of the plunger, it becomes possible to detect the leakage in the respective cases with reliability.

### BRIEF DESCRIPTION OF THE DRAWINGS

The above and other objects and advantages will be more apparent from the following detailed description of the preferred embodiments when read in connection with the accompanied drawings, wherein like reference numerals designate like or corresponding parts throughout the several views, and wherein:

FIG. 1 is a schematic diagram illustrating a biochemical analyzer according to an embodiment of the present invention;

FIG. 2 is a perspective diagram illustrating essential parts of a spot fixing device of the biochemical analyzer of FIG. 1;

FIG. 3 is a sectional diagram illustrating the spot fixing device as subjected to leak detection;

FIG. 4 is a perspective diagram illustrating the spot fixing device as subjected to leak detection;

FIG. 5 is a graph illustrating detection results in a normal condition of the spot fixing device;

FIG. 6 is a graph illustrating detection results in an abnormal condition where leakage is caused by defective sealing of a syringe of a syringe pump of the spot fixing device; and

FIG. 7 is a graph illustrating detection results in an abnormal condition where leakage is caused by a defect in a plunger of the syringe pump of the spot fixing device.

### DESCRIPTION OF THE PREFERRED EMBODIMENTS

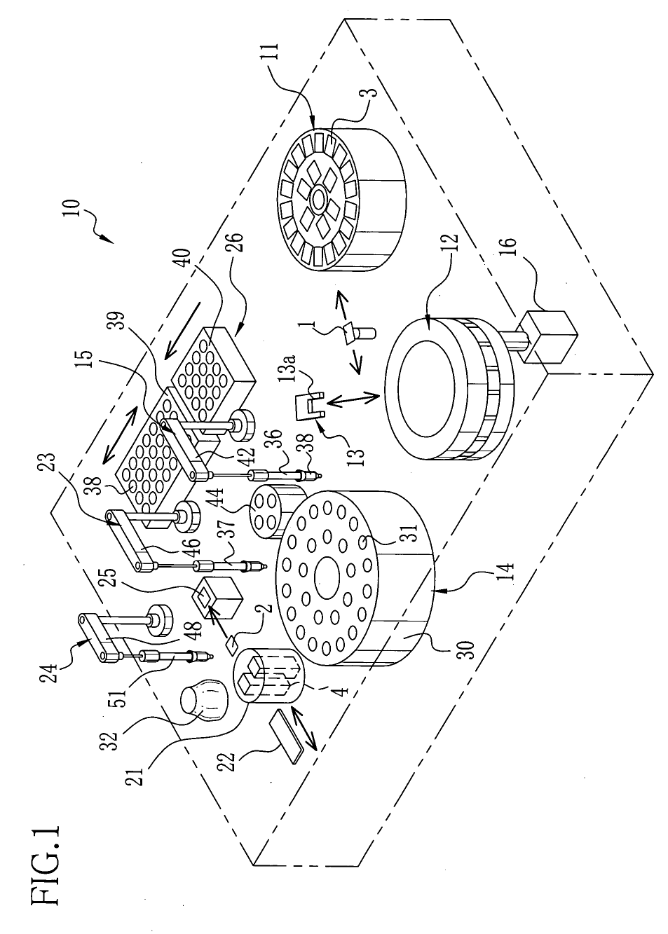

In FIG. 1, a biochemical analyzer 10 is provided with a main cartridge holder 11 holding a plural number of cartridges 3, each of which contains a dry type analyzing element 1 made of a dry type analyzing film chip, an incubator 12 disposed on one side of the main cartridge holder 11, an element carrier 13 for carrying the dry type analyzing element 1 from the main cartridge holder 11 to the incubator 12, a specimen holder 14 for holding sampling cups 31 containing specimens, for example, urine or serum, a first spot fixing device 15 for spotting the specimen as held in the specimen holder 14 onto the dry type analyzing element 1 while the dry type analyzing element 1 is carried by the element carrier 13 to the incubator 12, and a measuring device 16 disposed blow the incubator 12. The incubator 12 keeps the dry type analyzing element 1 at a constant temperature for a given time, to induce a color reaction of the specimen spotted on the dry type analyzing element 1. Thereafter, the measuring device 16 projects light onto the dry type analyzing element 1, to measure optical density of the specimen based on light reflected from or transmitted through the dry type analyzing element 1. Quantitative analysis is carried out based on the optical density. The main cartridge holder 11, the incubator 12, the element carrier 13, the specimen holder 14, the first spot fixing device 15 and the measuring device 16 constitute a colorimetric system or general measuring system.

The biochemical analyzer 10 is further provided with an electrolyte slide storage 21 storing electrolyte cartridges 4 containing electrolyte slides 2 as dry type analyzing elements for potentiometry, a second element carrier 22 for carrying the electrolyte dry type analyzing element 2 from the electrolyte cartridge 4 to a spot fixing position, a second spot fixing device 23 for spotting the specimen as held in the specimen holder 14 onto the electrolyte dry type analyzing element 2, a third spot fixing device 24 for spotting a referential fluid as held in a referential fluid container 32 onto the electrolyte dry type analyzing element 2, and a potentiometer 25 disposed in front of the spot fixing position for the electrolyte dry type analyzing element 2, for measuring a potential difference while keeping the electrolyte dry type analyzing elements 2 at a constant temperature for a given time. The electrolyte slide storage 21, the second element carrier 22, the second spot fixing device 23, the third spot fixing device 24 and the potentiometer 25 constitute a potentiometry system. Besides the first and second spot fixing devices 15 and 23, a tip supplier 26 is disposed, which has a tip rack 39 holding nozzle tips 38 in a sequence, wherein the nozzle tips 38 are formed as pipettes and are attached to tips of spotting nozzles 36 and 37 of the spot fixing devices 15 and 23.

The specimen holder 14 is provided with a rotary specimen table 30, which is loaded with a plural number of sampling cups 31 arranged in circles around its rotary center, so that the sampling cups 31 are placed in turn at a supply position.

The tip supplier 26 drives a diluting container 40, which is provided with an array of recesses to serve as cups, to slide in a lateral direction in addition to the tip rack 39. The movement of the tip supplier 26 is controlled such that the nozzle tip 38 or the recess of the diluting container 40 is placed underneath the nozzle 36 or 37 as they are placed above the tip supplier 26.

The first spot fixing device 15 has the spotting nozzle 36 at a tip of a sampling arm 42 that is mounted to be able to turn about and move up and down. The spotting nozzle 36 sucks the specimen from the sampling cup 31 on the rotary specimen table 30, moves to a spot fixing position to drip and fix a spot of the specimen on the dry type analyzing element 1 that is held on a carriage member 13a of the element carrier 13. A diluting fluid holder 44 is located beside the rotary specimen table 30, permitting diluting the specimen in the diluting container 40 before dripping it on the dry type analyzing element 1, according to measurement items. The structure of the sampling arm 42 will be concretely described later. After each spot fixing, the nozzle tip 38 is changed and thrown away in a not-shown disposal box.

The second spot fixing device 23 also has a sampling arm 46 like the sampling arm 42, which is mounted to be able to turn about and move up and down, and has at its tip the spotting nozzle 37 that sucks and discharges the specimen, and the nozzle tip 38 is attached to a tip of each spot fixing nozzle 37, to suck the specimen from the sampling cup 31 on the rotary specimen table 30, and move to the spot fixing position to drip and fix a spot of the specimen on the electrolyte dry type analyzing element 2.

The third spot fixing device 24 also has an arm 48 that is mounted to be able to turn about and move up and down. The arm 48 holds at its one end a spotting nozzle 51 that sucks the referential fluid from the referential fluid container 32 and drips it to fix a spot of the referential fluid on the electrolyte dry type analyzing element 2. The potentiometer 25 measures a potential difference between the specimen and the referential fluid on the electrolyte dry analysis elements 2. After being used, the electrolyte dry analysis elements 2 are thrown away in a not-shown disposal box.

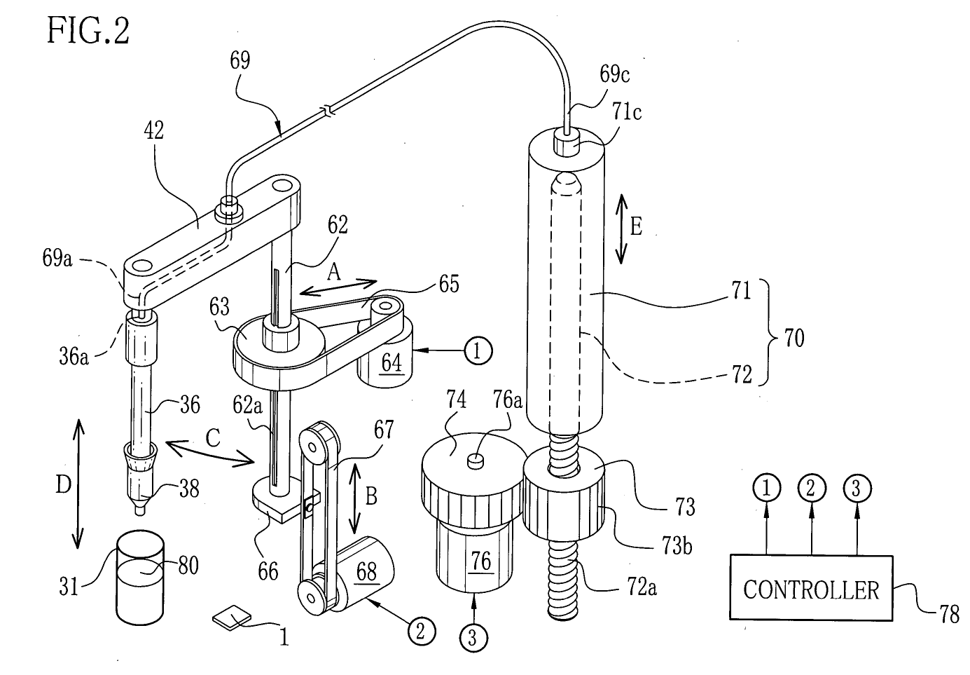

The first spot fixing device 15 is configured as shown in detail in FIG. 2. The first spot fixing device 15 is constituted of the spotting nozzle 36, the sampling arm 42, a spline shaft 62, a belt wheel 63, a belt 65, a first motor 64, a flange 66, a belt 67, a second motor 68, an air pipe 69, a syringe pump 70, a transmission member 73, a drive gear 74 and a third motor 76.

The sampling arm 42 holds at its first end the spotting nozzle 36 to be movable up and down. The spline shaft 62 is oriented vertical, supporting the sampling arm 42 at its second end to keep it horizontally. The belt wheel 63 is provided with a hook that is engaged in a groove 62a formed in a peripheral wall of the spline shaft 62 along its axial direction, so that the belt wheel 63 is fixed in a rotational direction but is movable in the axial direction relative to the spline shaft 62. One end of the belt 65 is suspended around the belt wheel 63. The first motor 64 drives the belt 65 to move in a direction of an arrow A, to rotate the belt wheel 63 in a given direction by a given amount. The flange 66 is mounted to a bottom end of the spline shaft 62, and the belt 67 is affixed to a side wall of the flange 66 by a screw. The belt 67 is moved by the second motor 68 in a direction of an arrow B, to move the spline shaft 62 a given amount up or down.

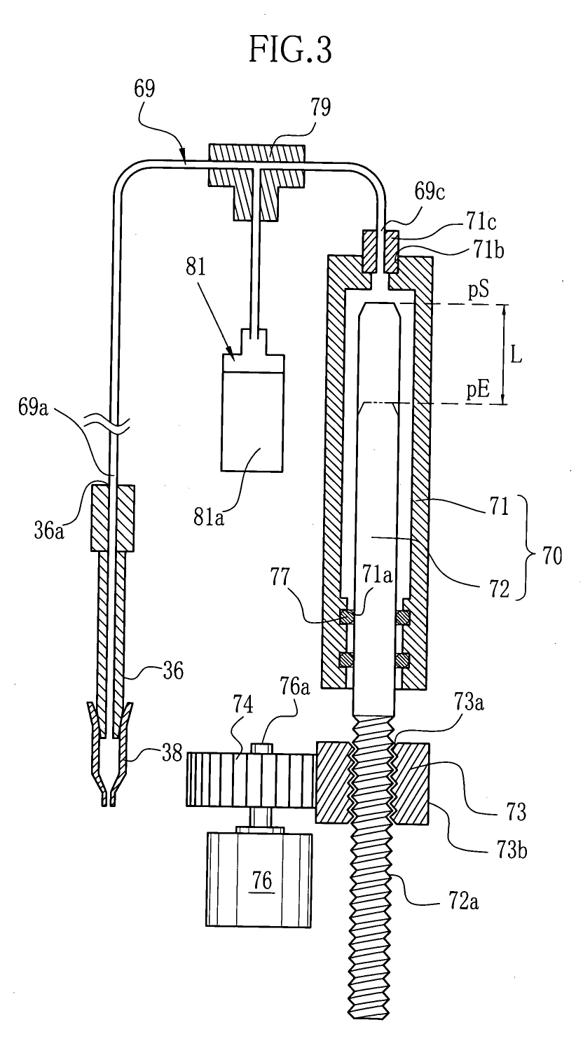

The air pipe 69 is mounted in the sampling arm 42, with one end 69a connected to one end 36a of the spotting nozzle 36, which protrudes toward the first end of the sampling arm 42. The air pipe 69 extends outside from a position near the second end of the sampling arm 42. A second end 69c of the air pipe 69 is connected to a vent port 71b of the syringe pump 70 through a connection rod 71c, as shown in FIG. 3.

The syringe pump 70 consists of a syringe that is shaped into a cylindrical body and is connected to the second end 69c of the air pipe 69, and a plunger 72 that is fitted in the syringe 71 to be movable up and down and has male helicoids 72a formed around its external peripheral portion that protrudes out of the syringe 71. The syringe 71 also has a groove 71a formed in its internal periphery along a circumferential direction, and a sealing member 77 is fitted in the groove 71a to keep air-tightness between the syringe pump 70 and the syringe 71. The transmission member 73 has female helicoids 73a formed around its internal periphery, to mesh with the male helicoids 72a of the plunger 72. The transmission member 73 has a gear 73b formed around its external periphery, and the gear 73b gears into the drive gear 74 that is fixedly mounted to a rotary shaft 76a of the third motor 76.

As the third motor 76 rotates, the rotational movement is transmitted through the transmission member 73 to the plunger 72, to drive the plunger 72 to move up or down by a given amount according to leads of the male and female helicoids 72a and 74a. As a result, an internal volume of the syringe 71 changes to send the air into or suck the air from the air pipe 69 through the second end 69c, so that a pressure is supplied to a pipe system consisting of the nozzle tip 38, the spotting nozzle 36 and the air pipe 69.

The first to third motors 64, 68 and 76 of the first spot fixing device 15 are connected to a controller 78 that controls the motors 64, 68 and 76 according to predetermined sequence programs.

First, the controller 78 outputs a motor drive signal to the first motor 64, causing the sampling arm 42 to rotate in a direction of an arrow C (see FIG. 2) by the driving power of the first motor 64 that rotates according to the motor drive signal, so as to position the spotting nozzle 36 and the nozzle tip 38 above the sampling cup 31 that contains a specimen 80.

Next, the controller 78 drives the third motor 76 in a forward direction to move the plunger 72 upward. Thereby the air is sent from the syringe 71 into the air pipe 69, so the pipe system is positively pressured. Simultaneously, the controller 78 drives the second motor 68 to rotate in a direction to move the sampling arm 42 downward at a given speed. When the sampling arm 42 moves down to bring a tip of the nozzle tip 38 into contact with a liquid surface of the specimen 80 in the sampling cup 31, a not-shown liquid surface detection unit detects it, so the controller 78 stops driving the second motor 68 to stop downward movement of the sampling arm 42. Simultaneously, the controller 78 stops driving the third motor 76 to stop upward movement of the plunger 72 and thus stop sending the air from the syringe 71 into the air pipe 69.

Thereafter, the controller 78 drives the third motor 76 to rotate reversely to move the plunger 72 downward by a given amount. Thereby, the specimen 80 is sucked by a predetermined amount into the nozzle tip 38. Then, the controller 78 outputs drive signals sequentially to the respective motors 64, 68 and 76, to move the sampling arm 42 upward and rotate it by a given angle and then move it downward, so that the specimen 80 sucked in the nozzle tip 38 is discharged from the nozzle tip 38 and is fixed as a spot on the dry type analyzing element 1. According to the present embodiment, the specimen is discharged 5 ml to 100 ml at a time. The second and third spot fixing devices 23 and 24 have the same structure as the first spot fixing device 15.

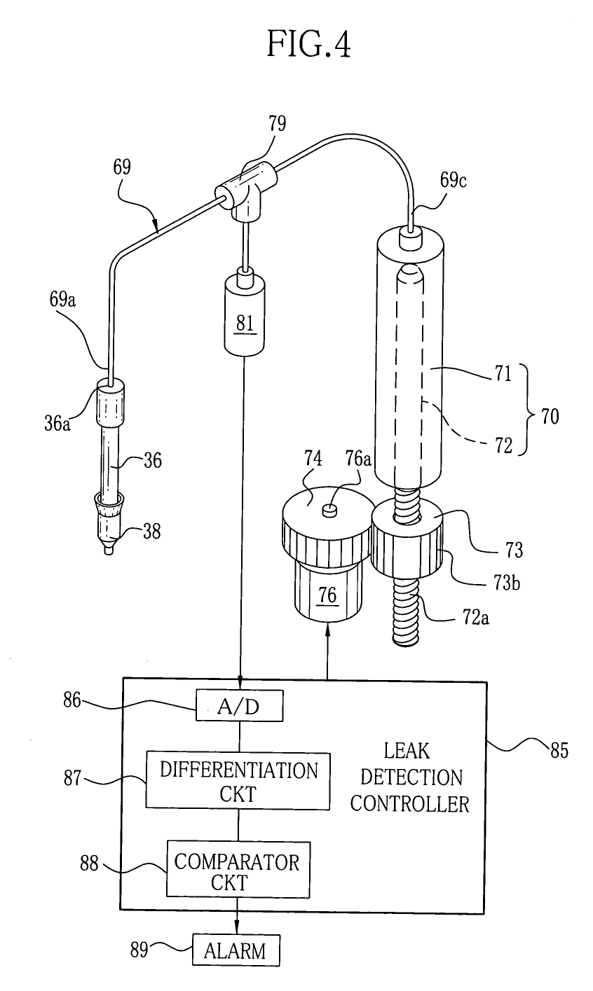

According to the present embodiment, the spot fixing devices 15, 23 and 24 are subjected to a leak test before the biochemical analyzer 10 is shipped as a product. Now the leak test will be described with respect the spot fixing device 15, as shown in FIGS. 3 and 4, and the same applies to the other spot fixing devices 23 and 24. For the leak test, the spotting nozzle 36 is connected to the syringe pump 70 through the air pipe 69, and the air pipe 69 is connected at its intermediate position to a pressure sensor 81 through a three-way pipe 79. The syringe pump 70 is also connected to the transmission member 73, the drive gear 74 and the third motor 76, in the same way as described above with respect to the first spot fixing device 15. The pressure sensor 81 and the third motor 76 are connected to a leak detection controller 85.

The pressure sensor 81 outputs electric detection signals representative of pressure values inside the air pipe 69, and sends them to the leak detection controller 85. The pressure sensor 81 is provided with a piezoelectric element 81a that generates piezoelectricity corresponding to the internal pressure of the air pipe 69, so the current generated from the piezoelectric element 81a is output as the detection signal. The leak detection controller 85 is provided wit an A/D converter 86, a differentiation circuit 87 and a comparator circuit 88. The A/D converter 86 digitalizes the analog electric signal from the pressure sensor 81. The differentiation circuit 87 differentiates the digitalized electric signal, to clearly show variations in pressure as derivative values of the pressure. The comparator circuit 88 is fed with a predetermined threshold value as a reference signal, to judge from the comparison with the threshold value as to whether any leakage takes place or not. The threshold value may vary depending upon what kind of leakage should be detected, using not-shown operational members like a keyboard. The differentiation circuit 87 and the comparator circuit 88 may be embodied as hardware devices but may preferably embodied as software programs installed and executed in the leak detection controller 85. If the comparator circuit 88 detects a leakage as the pressure derivative value goes above the threshold value, the leak detection controller 85 lets an alarm 89 go off. As a device for warning the leakage, a not-shown display device may display a warning instead of or in addition to the alarm 89.

In the leak test of the present embodiment, the plunger 72 of the syringe pump 70 is moved up and down in the same work range L and at the same speed as for its actual sucking discharging operation, to output the internal pressure of the air pipe 69 through the pressure sensor 81, and detect the pressure derivative values through the differentiation circuit 87. More specifically, the syringe pump 70 moves from a lowermost position pE as shown by phantom lines in FIG. 3, where the plunger 72 is pulled out farthest from the syringe 71, to an uppermost position pS as shown by solid lines in FIG. 3 where an inner tip of the plunger 72 is pushed up to a top end of the syringe 71. Continuously to the upward movement, the plunger 72 is moved from the uppermost position pS down to the lowermost position pE. The internal pressure is detected during the reciprocation of the plunger 72. It is to be noted that the tip of the spotting nozzle 36 is not closed but kept open during the leak detection.

As described so far, the spotting nozzle 36 and the air pipe 69 constitute an open pipe system, and the pressure sensor 81 is connected to the open pipe system through the three-way pipe 79. The open pipe system, the pressure sensor 81 and the leak detection controller 85 constitute a leak detection unit. The open pipe system, the pressure sensor 81 and the differentiation circuit of the leak detection controller 85 are shared with the above-mentioned not-shown liquid surface detection unit, so as to simplify the apparatus structure.

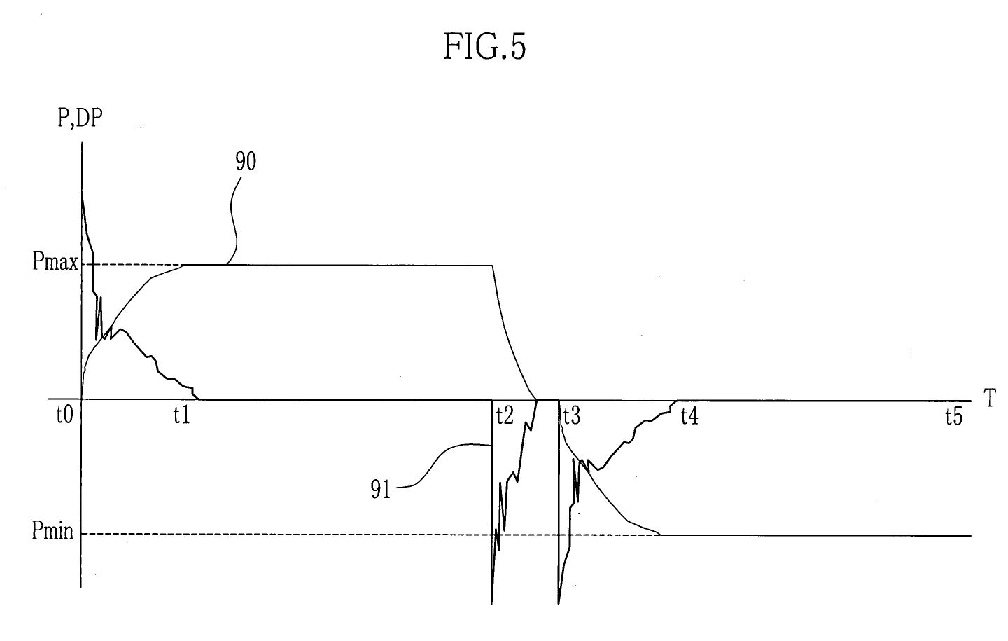

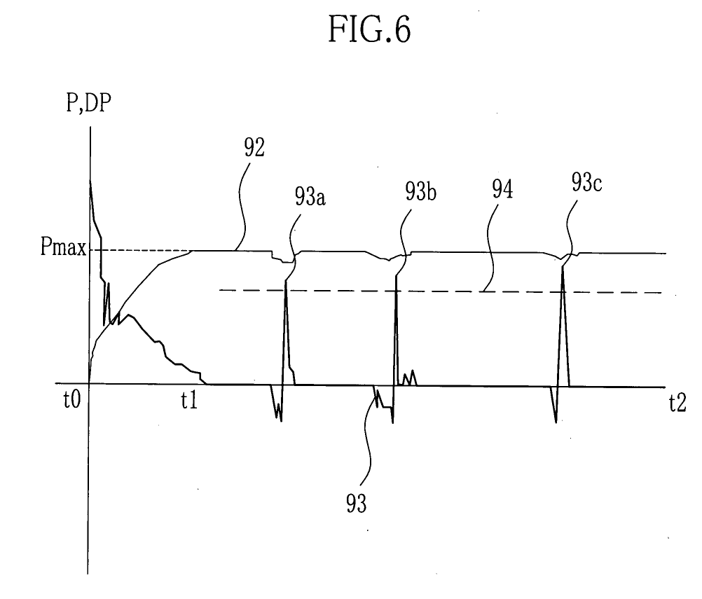

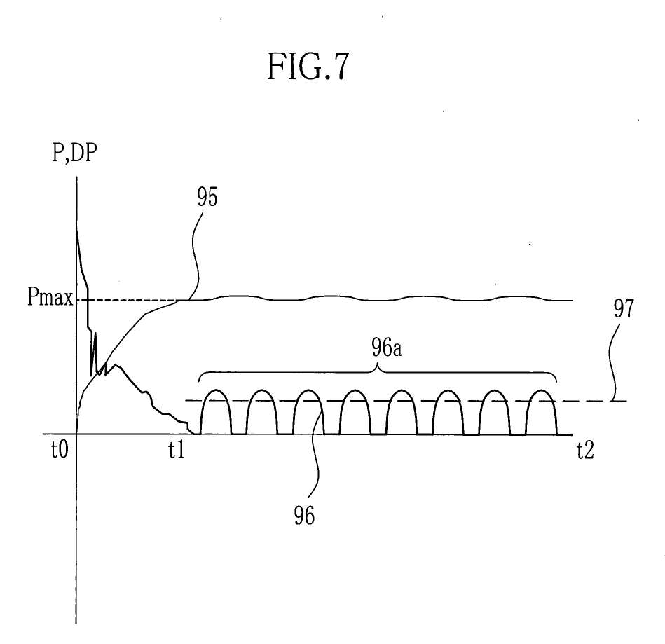

FIGS. 5 to 7 show results of the leak test on three cases, wherein FIG. 5 shows a curve 90 of the internal pressure P and a curve 91 of the derivative values DP of the pressure in a normal case without any leakage, FIG. 6 shows curves 92 and 93 of the internal pressure P and derivative values DP of the pressure in a case where a leakage occurs due to defective sealing of the syringe 71 to the plunger 72, and FIG. 7 shows curves 95 and 96 of the pressure P and derivative values DP of the pressure in a case where a leakage occurs due to a defection of the plunger 72.

In these graphs, the horizontal axis represents time T, and the plunger 72 is located in the lowermost position pE at a measurement starting time t0. From this point, the third motor 76 starts rotating to move the plunger 72 upward. As the plunger 72 moves upward, the internal pressure P increases gradually and reaches a maximum value Pmax at a time point t1. From the measurement starting time t0 to the time point t1, the derivative value DP gradually decreases, which means that the pressure P fluctuates lesser.

In the normal condition, the pressure P is kept at the maximum value Pmax from the time point t1 till the plunger 72 comes to the uppermost position pS at a time point t2, as shown by the curve 90. While the pressure P is kept unchanged, that is, from the time point t1 to the time point t2, the derivative value DP is kept at zero, as shown by the curve 91. From the time point t2 to a time point t3, the motor 76 stops, so the plunger 72 stays in the uppermost position pS. During this time period, the pressure P decreases gradually and comes to zero at the time point t3. At the time point t2, the derivative value DP becomes negative, and then comes back to zero at the time point t3. Since the plunger 72 stops in the time period from t2 to t3, the derivative values DP detected during this time period are not used for the leak detection.

From the time point t3, the motor 76 starts rotating reversely to move the plunger 72 downward. From the time point t3 to a time point t4, the pressure P decreases gradually, that is, the negative pressure increases gradually, and the pressure P reaches a minimum value Pmin, i.e. a maximum negative pressure value, at the time point t4. From the time point t4 till the plunger 72 comes to the lowermost position pE at a time point t5, the pressure P is kept at the minimum value Pmin. When the plunger 72 comes to the lowermost position pE, one cycle of the leak test is accomplished. As shown by the curve 91, the derivative value DP becomes negative at the time point t3, and then increases gradually till the time point t4. From the time point t4 to the time point t5, the derivative value DP is kept zero.

As described so far, if the first spot fixing device 15 is in the normal condition, the pressure P detected by the pressure sensor 81 little fluctuates in most time during the reciprocation of the plunger 72 except the time periods immediately after the plunger 72 starts moving in either direction, so the pressure derivative value DP is mostly kept zero. In this condition, the first spot fixing device 15 can suck and discharge a predetermined amount of liquid exactly. As obvious from the graph of FIG. 5, the pressure P and the derivative value DP vary with the movement of the plunger 72 in a symmetrical fashion between the time period from t0 to t2, that is, while the plunger 72 moves from the lowermost position pE up to the uppermost position pS, and the time period from t3 to t5, that is, while the plunger 72 moves from the uppermost position pS down to the lowermost position pE, although they are positive on one hand, and are negative on the other hand. So the result of leak judgment derived from the curves will be substantially same in both periods. Therefore, FIGS. 6 and 7 show merely the results of measurement during the upward movement of the plunger 72, i.e. from the lowermost position pE to the uppermost position pS.

As shown in FIG. 6, if the sealing of the syringe 71 is not good, the derivative value DP indicated by the curve 93 fluctuates greatly at several points as designated by 93a, 93b and 93c, during the time period from the time point t1 when the pressure P indicated by the curve 92 reaches the maximum value Pmax to the time point t2 when the plunger 72 comes to the uppermost position pS. According to the present embodiment, a threshold value is predetermined for detecting the leakage due to the defective sealing of the syringe 71, as shown by dashed lines 94 in FIG. 6. The threshold value 94 is fed as a reference signal to the comparator circuit 88, so the pressure derivative value DP from the differentiation circuit 87 is compared with the threshold value 94. If the pressure derivative value DP is above the threshold value 94, the leak detection controller 85 judges the sealing member 77 to be defective, and lets the alarm 89 go off.

If on the other hand the leakage occurs because the plunger 72 is defective, the pressure derivative value DP indicated by the curve 96 in FIG. 7 varies at regular intervals in a period 96a from the time point t1 when the pressure P indicated by the curve 92 reaches the maximum value Pmax to the time point t2 when the plunger 72 comes to the uppermost position pS. This variation patterns of the derivative values DP indicate that the leakage occurs at the regular intervals because the plunger 72 leans to an axial direction of the syringe 71, or because the plunger 72 is defective with respect to roundness. In order to detect the leakage due to the defect in the plunger 72, a second threshold value 97 is predetermined, as shown by dashed lines in FIG. 7. The threshold value 97 is fed as a reference signal to the comparator circuit 88, so the pressure derivative value DP from the differentiation circuit 87 is compared with the threshold value 97. If the pressure derivative value DP is above the threshold value 97, the leak detection controller 85 judges the plunger 72 to be defective.

As described so far, since the derivative value of the pressure is used for detecting the leakage, it is possible to detect small variations in pressure that is measured by the pressure sensor 81, so the leakage can be detected with high accuracy whichever the leakage is caused by defect in the syringe 71 or the plunger 72 of the syringe pump 70.

Because the malfunctions of the syringe pump 70 are detected based on the derivative value of the pressure that is measured by the pressure sensor 81 while moving the plunger 72 of the syringe pump 70 in the same work range L as in the actual sucking discharging operation, it becomes possible to detect such malfunctions without fail that can occur during the actual sucking discharging operation. Furthermore, because the leak detection is carried out with the tip of the spotting nozzle 36 open, the pressure is measured under proximate conditions to the actual sucking discharging operation, so it is possible to judge the malfunctions of the syringe pump 70 with reliability.

Although the leak detection of the syringe pump 70 is carried out before the shipment of the biochemical analyzer 10 from the factory in the above described embodiment, it is possible to carry out the same leak detection as above after the shipment of the biochemical analyzer 10, before the actual use thereof.

The present invention is not to be limited to the above embodiments but, on the contrary, various modifications will be possible without departing from the scope of claims appended hereto.

What is claimed is:

1. A method of detecting disorder of a syringe pump comprising a cylindrical syringe and a plunger sliding inside said syringe, said method comprising steps of:
connecting an open pipe system to a vent port of said syringe;
measuring pressure in said open pipe system while moving said plunger relative to said syringe;
obtaining derivative values of the measured pressure; and
judging disorder of said syringe pump based on said pressure derivative values.

2. A method of detecting disorder of a syringe pump as claimed in claim 1, wherein pressure of said open pipe system is measured while moving said plunger relative to said syringe in a working range used in an actual operation of said syringe pump.

3. A method of detecting disorder of a syringe pump as claimed in claim 1, wherein disorder of said syringe pump is judged by comparing said pressure derivative values with predetermined threshold values.

4. A method of detecting disorder of a syringe pump comprising a cylindrical syringe and a plunger sliding inside said syringe, said syringe pump being connected to a spot fixing nozzle to supply sucking and discharging pressure to said spot fixing nozzle for causing said spot fixing nozzle to suck and discharge a liquid through its tip or through a nozzle tip attached to the tip of said spot fixing nozzle, to fix a spot of said liquid on a sampling material, said method comprising steps of:
connecting a pressure sensor to a pressure system that is connected between said spot fixing nozzle and said syringe pump;
obtaining pressure derivative values by differentiating pressure signals output from said pressure sensor while said plunger is being moved relative to said syringe; and
judging disorder of said syringe pump based on said pressure derivative values.

5. A method of detecting disorder of a syringe pump as claimed in claim 4, wherein said pressure sensor outputs the pressure signal while said plunger is being moved relative to said syringe, with the tip of said spot fixing nozzle open.

6. A method of detecting disorder of a syringe pump as claimed in claim 4, wherein said pressure sensor outputs the pressure signal while said plunger is being moved relative to said syringe in a working range used in an actual operation of said syringe pump.

7. A method of detecting disorder of a syringe pump as claimed in claim 4, wherein disorder of said syringe pump is judged by comparing said pressure derivative values with predetermined threshold values.

8. A liquid sucking discharging device comprising:
a syringe pump comprising a cylindrical syringe and a plunger sliding inside said syringe;
an open pipe system connected to a vent port of said syringe;
a pressure sensor for measuring pressure in said open pipe system;
a differentiation device for differentiating pressure signals output from said pressure sensor; and
a judging device for judging disorder of said syringe pump base on pressure derivative values obtained by said differentiation device.

9. A liquid sucking discharging device as claimed in claim 8, wherein said open pipe system comprises a nozzle that sucks and discharges a liquid, and a pipe connecting said nozzle to the vent port of said syringe pump.

10. A liquid sucking discharging device as claimed in claim 8, wherein said pressure sensor detects the pressure in said open pipe system while said plunger is being moved relative to said syringe in a working range used in an actual operation of said syringe pump, with the tip of said nozzle open.

11. A liquid sucking discharging device as claimed in claim 8, wherein said judging device judges disorder of said syringe pump by comparing said pressure derivative values with predetermined threshold values.

12. A liquid sucking discharging device comprising:
a spot fixing nozzle that sucks and discharges a liquid through its tip or through a nozzle tip attached to the tip of said spot fixing nozzle, to fix a spot of said liquid on a sampling material;
a syringe pump connected to said spot fixing nozzle to supply sucking and discharging pressure to said spot fixing nozzle, said syringe pump comprising a cylindrical syringe and a plunger sliding inside said syringe;
a pressure sensor connected to a pressure system that is connected between said spot fixing nozzle and said syringe pump;
a differentiation device for differentiating pressure signals output from said pressure sensor, to obtain pressure derivative values;
a judging device for judging disorder of said syringe pump base on said pressure derivative values.

13. A liquid sucking discharging device as claimed in claim 12, wherein said pressure sensor outputs the pressure signals while said plunger is being moved relative to said syringe in a working range used in an actual operation of said syringe pump, with the tip of said spot fixing nozzle open.

14. A liquid sucking discharging device as claimed in claim 8, wherein said judging device judges disorder of said syringe pump by comparing said pressure derivative values with predetermined threshold values.

15. A biochemical analyzer comprising:
a spot fixing nozzle that sucks and discharges a liquid through its tip or through a nozzle tip attached to the tip of said spot fixing nozzle, to fix a spot of said liquid on a sampling material;
a syringe pump connected to said spot fixing nozzle to supply sucking and discharging pressure to said spot fixing nozzle, said syringe pump comprising a cylindrical syringe and a plunger sliding inside said syringe;
a measuring device that projects measuring light onto said sampling material to measure light reflected from or transmitted through said sampling material, and makes quantitative analysis based on the results of measurement;
a pressure sensor connected to a pressure system that is connected between said spot fixing nozzle and said syringe pump;
a differentiation device for differentiating pressure signals output from said pressure sensor, to obtain pressure derivative values;
a judging device for judging disorder of said syringe pump base on said pressure derivative values.

16. A biochemical analyzer as claimed in claim 15, wherein said pressure sensor outputs the pressure signals while said plunger is being moved relative to said syringe in a working range used in an actual operation of said syringe pump, with the tip of said spot fixing nozzle open, and said judging device judges disorder of said syringe pump by comparing said pressure derivative values with predetermined threshold values.

17. A biochemical analyzer as claimed in claim 16, further comprising a warning device that goes off a warning when said syringe pump is judged to be disordered.
